(19) United States
(12) Patent Application Publication
(10) Pub. No.: US 2020/0395122 A1
(43) Pub. Date: Dec. 17, 2020

(54) HEALTH MANAGEMENT SYSTEM AND PROGRAM FOR SAME

(71) Applicant: AQUA BANK CO.,LTD., Osaka-shi, Osaka (JP)

(72) Inventor: Takashi TAKEHARA, Osaka-shi, Osaka (JP)

(21) Appl. No.: 16/761,448

(22) PCT Filed: Nov. 5, 2018
(86) PCT No.: PCT/JP2018/041058

§ 371 Date: May 4, 2020

(30) Foreign Application Priority Data
Nov. 9, 2017 (JP) 2017-216613
Oct. 22, 2018 (JP) 2018-198737

(51) Int. Cl. G16H 40/67; G16H 40/40; G16H 50/20; A61M 16/12; A61B 5/00

(52) U.S. Cl. CPC G16H 40/67; G16H 40/40; G16H 50/20; G16H 10/60; A61B 5/6801; A61B 5/0002; A61M 16/12

(57) ABSTRACT

A healthcare system capable of ingesting hydrogen, oxygen, or the like required based on a vital sign of a user by ingestion with a portable gas generator, whereby the healthcare system can ingest an appropriate gas according to the physical condition of the user. The healthcare system includes: a portable gas generator capable of orally suctioning a gas selected from hydrogen and/or a mixed gas of a gas having an effect of promoting neural activity and/or blood circulation activity of an oxygen-containing living body, the gas being produced by electrolysis of an electrolyte filled in an internal electrolysis tank of the healthcare system; and a wearable portable terminal capable of detecting a vital sign that has been fixed in contact with a part of a human body part and has been digitized.

### TECHNICAL FIELD

The present invention relates to a health management system and a program for use in the health management system, which are capable of taking an appropriate gas according to a physical condition of a user by taking hydrogen, oxygen, or the like, which is required based on a vital sign of the user detected by a wearable portable terminal, orally by a portable gas generator.

### THE BACKGROUND OF THE INVENTION

In recent years, the efficacy of hydrogen has attracted attention in studies on various animal diseases such as neurodegenerative diseases and acute pulmonary disorders, and human clinical studies in metabolic syndromes and diabetes mellitus, and various studies on medical applications have been actively conducted. Hydrogen is said to be effective in removing only the bad reactive oxides (=hydroxyl radicals), which are responsible for accelerating aging and causing various diseases such as arteriosclerosis and carcinoma, from the body. Since it does not adversely affect tissues and cells of the body, there are a wide range of methods for taking it into the body, such as intravenous administration, oral administration of aqueous solution, and inhalation of gas.

It is recommended to introduce hydrogen into the body to prevent aging and to promote beauty and well-being in a variety of conditions, particularly during exercise, stress, drinking, smoking, staying in ultraviolet/contaminated environments, sleeping deficiencies, long working hours, etc., when active O2 is likely to occur in the body. In particular, the Applicant has provided the results of the study focusing not only on the daily-aged effects due to hydrogen consumption but also on the immediate and short-time mental-physical effects due to gas aspiration of hydrogen, which had not been clarified in the past (see Patent Document 1).

Moreover, oxygen is used for generating energy of a cell and is an indispensable element for metabolism of the human body. Attention has been paid to activation of the cells in the body by oxygen, and studies have been made in recent years that conscious intake of oxygen into the body is effective in promotion of natural healing of disease conditions such as fatigue recovery and fracture, improvement of hematogenous disorder, beauty, stress reduction and the like. Actually, it is known that athletes use oxygen capsules for body shaping or treatment of injuries, and oxygen masks are used for patients with weakened physical strength.

In view of such circumstances, in recent years, there has been provided a gas generator such as a hydrogen which can consume hydrogen and oxygen, and the applicant has also provided a gas generator which is rechargeable, small in size and inexpensive so that the gas generator can be carried by a user and can be carried freely, and further, a gas generator which can selectively generate hydrogen and oxygen has been provided.

### ART DISCUSSED ABOVE

### Patented Literature

[Patent Document 1] WO2018/151107 of the International Publication

### DISCLOSURE OF INVENTION

### Problems to be Solved by the Invention

However, conventional hydrogen and other generators consume hydrogen and the like only by themselves, and because the user manages his/her physical condition and consumes hydrogen and the like at his/her own discretion when desired, it is not always possible to consume an appropriate amount of hydrogen and the like when required, and it is incomplete as a health-promoting tool. On the other hand, in recent years, with the development of wearable terminals, basic vital signs such as heart rate and respiration rate can be routinely measured, and it is easy to diagnose the physical condition of the user in real time.

The present invention has been made in view of the above circumstances, and it is an object of the present invention to provide a health management system and programs used in the health management system, which can ingest hydrogen, oxygen, or the like required based on a vital sign of a user by ingestion with a portable gas generator, thereby taking an appropriate gas according to the physical condition of the user.

### Means for Solving the Problem

In order to solve the above-mentioned problems, the present invention provides a healthcare system comprising:

a portable gas generator capable of suctioning a selected gas among mixed gases containing hydrogen and/or oxygen, which are produced by electrolysis of an electrolyte filled in at least an electrolysis tank provided inside, for a predetermined period of time by spontaneous respiration, wherein said mixed gas having an effect of promoting neural activity and/or circulatory activity of a living body; and

a wearable portable terminal for detecting a vital sign that is digitized by contacting and fixing a part of a body part.

According to the present invention, it is possible to take appropriate gas according to the physical condition of the user by taking hydrogen, oxygen, or the like required based on the vital signs of the user detected by the wearable portable terminal by ingestion with a portable gas generator.

Further, the program used in the healthcare system includes a detecting means for detecting the vital signs that are digitized by contacting and fixing the wearable portable terminal to a part of a human body part;

a first wireless transmitting means for wirelessly transmitting the vital signs to the outside; and

a calculating means for calculating a required gas and gas discharge amount and/or time that are set in advance in accordance with each of the vital signs detected by the wearable portable terminal, and further comprising any one of the following (1), (2) and (3).

(1) Second wireless transmission means for wirelessly transmitting the required gas discharged from the gas generator and the gas discharge amount and/or time to the outside
(2) Control signal transmitting means for transmitting a control signal based on the calculated required gas and gas discharge amount and/or time, and control means for receiving the control signal to control the power supply of the portable gas generator
(3) Second wireless transmission means for wirelessly transmitting the required gas and the gas discharge amount and/or time discharged from the gas generator to the outside, control signal transmission means for transmitting a control signal based on the calculated required gas and gas discharge amount and/or time, and control means for receiving the control signal and controlling the power supply of the portable gas generator

According to the present invention, data of a vital sign of a user detected by a wearable portable terminal is transmitted to a portable terminal such as a smart phone or a server, for example, and is analyzed by an installed analysis application (calculation means) to detect a selection of a gas necessary for the user, a necessary amount, and the like. This allows the user to ingest a gas corresponding to his/her physical condition with a portable gas generator.

In addition, the user can automatically ingest a necessary amount of gas by transmitting a control signal for operating the portable gas generator with respect to the necessary gas in real time, which is calculated by the portable communication terminal. Therefore, the user can ingest the optimum gas and manage the health without making a self-judgment.

Here, as in (1), when only the second wireless transmission means is provided, the user can manually select a gas of a desired type, amount, and time using the analysis result as an index, and ingest the gas from the portable gas generator.

As shown in (2), when only the control signal transmitting means and the control means is provided, the user automatically selects the desired type, amount, and time of the gas in the index of the analysis result, at this time, it may be manual only the start and end of the generation of the gas.

When all means of the second radio transmission means, the control signal transmission means, and the control means are provided as in (3), either automatic or manual can be adopted.

The wearable portable terminal may include the calculating means. That is, the selection and calculation of the necessary gas may be performed by the wearable portable terminal. Specifically, the wearable portable terminal includes calculation means for calculating a necessary gas and a gas discharge amount and/or time set in advance in accordance with each of the detected vital signs.

Further, the wearable portable terminal at this time transmits a control signal based on the calculated required gas and gas emission (discharge) amount and/or time, control board of the portable gas generator receives the control signal, it is also possible to control the power supply.

According to the present invention, since a portable terminal such as a smartphone or a server is unnecessary and the system can be implemented only with a wearable portable terminal and a portable gas generator, the user can select whether to perform calculation via a portable terminal such as a smartphone or a server or perform calculation in a wearable portable terminal in accordance with the situation such as the degree of satisfaction of the radio transmission situation. Therefore, it can be said that the use of the system in this case is widened.

Incidentally, the above portable gas generator as an example,

a body cover member having a battery, a control board for controlling power supply from the battery; and a pair of anode electrodes energized or blocked an anode and cathode of battery by the control board;

a water reservoir electrolysis tank detachably attached to the body cover member, the pair of anode electrodes are inserted into the interior in a mounted state;

a nozzle portion having a through hole; and

a mixing portion having a flow path for fluidly connecting nozzle portion and an end portion of the electrolysis tank and introducing atmosphere.

### Effect of the Invention

According to the portable gas generator of the present invention, the gas can be ingested appropriately according to the physical condition of the user by ingesting hydrogen, oxygen, or the like, which is required based on the vital signs of the user detected by the wearable portable terminal, by the portable gas generator of the present invention, and the health promotion and prevention/improvement of the symptoms of the individual user can be carried out by using the portable gas generator of the present invention.

### BRIEF DESCRIPTION OF THE DRAWINGS

FIG. 1 It is an exploded view of an assembly illustrating for each member of the gas generator of the present invention.

FIG. 2 shows a view from each direction of the gas generator of the present invention of FIG. 1, (a) is a left side view, (b) is a front view, (c) is a right side view, (d) is a bottom view, (e) shows a top view.

FIG. 3 The gas generator of the present invention of FIGS. 1-2 shows a cross-sectional view taken along line A-A of FIG. 2 (c).

FIG. 4 It is a diagram showing an outline of a health management system of the present invention.

FIG. 5(a) FIG. 1 is a diagram showing a schematic diagram of a program of an embodiment of a health management system of the present invention;

FIG. 5(b) It is a diagram showing a schematic view of a program of another embodiment of the health management system of the present invention.

FIG. 5(c) It is a diagram showing a schematic view of a program of another embodiment of the health management system of the present invention.

FIG. 6(a) It is a diagram showing a program flow of the health management system of the present invention.

FIG. 6(b) It is a diagram showing a program flow of the health management system of the present invention.

FIG. 6(c) It is a diagram showing a program flow of the health management system of the present invention.

FIG. 6(d) It is a diagram showing a program flow of the health management system of the present invention.

FIG. 7(a) It is a diagram showing a flow of a calculation program of the present health management system of the present invention.

FIG. 7(b) It is a diagram showing a flow of a calculation program of the present health management system of the present invention.

FIG. 7(c) It is a diagram showing a flow of a calculation program of the present health management system of the present invention.

FIG. 7(d) It is a schematic diagram showing a method of setting the required gas based on the setting information such as each vital sign.

FIG. 8 Illustration illustrating an example of the effects of using the present health management system of the present invention.

FIG. 9 It is a diagram showing an example of use of the present health management system of the present invention.

FIG. 10 It is a diagram showing another embodiment of the present health management system of the present invention.

FIG. 11 It is a diagram showing another embodiment of the present health management system of the present invention.

FIG. 12 Measurement graphs showing the average miosis (CR-value) of the left and right eyes of subjects before and after hydrogen aspiration are shown.

FIG. 13 A graphical representation of the temperature rise of forefinger skin (° C.) evaluation separately before and after hydrogen aspiration of the subject is shown as a graphical representation of the temperature rise of forefinger skin (° C.) shown before and after hydrogen aspiration.

FIG. 14 A graphical representation of the average before and after hydrogen aspiration of a subject in a cerebral stress assessment is shown.

FIG. 15 A graphical representation of the mean before and after hydrogen aspiration of a subject in assessing cerebral rotates is shown.

FIG. 16 A graphical representation of the subject's mean before and after hydrogen aspiration in assessing short-term memory and lateral cognitive function is shown.

FIG. 17 A graphical representation of the subject's mean before and after hydrogen aspiration in the results of the areal emotional status scale is shown.

### DETAILED DESCRIPTION OF THE INVENTION

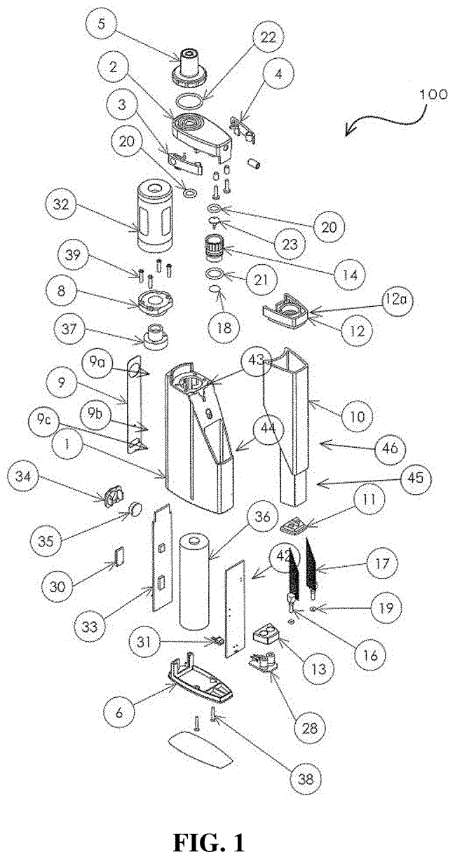

Hereinafter, a portable gas generator used in the health care system of the present invention will be exemplified. The results of tests to verify the psychological and physiological effects of hydrogen intake will be described later. First, an overview of the configuration of the portable gas generator will be described in FIGS. 1 to 3. It is needless to say that the portable gas generator is not limited to the one shown in the drawings, and the contents of the drawings and the description may be modified within the scope of general knowledge. Further, each of the drawings may be displayed by exaggerating dimensions, ratios and numbers as occasion demands for easy understanding.

The gas generator is characterized in that it comprises a partition member and opening/closing means which are the main components for separating the generated hydrogen and oxygen.

In explaining the present invention, for a simple understanding, first, the organization “excluding the partition member and the opening and closing means” will be described in detail with reference to FIGS. 1 to 3.

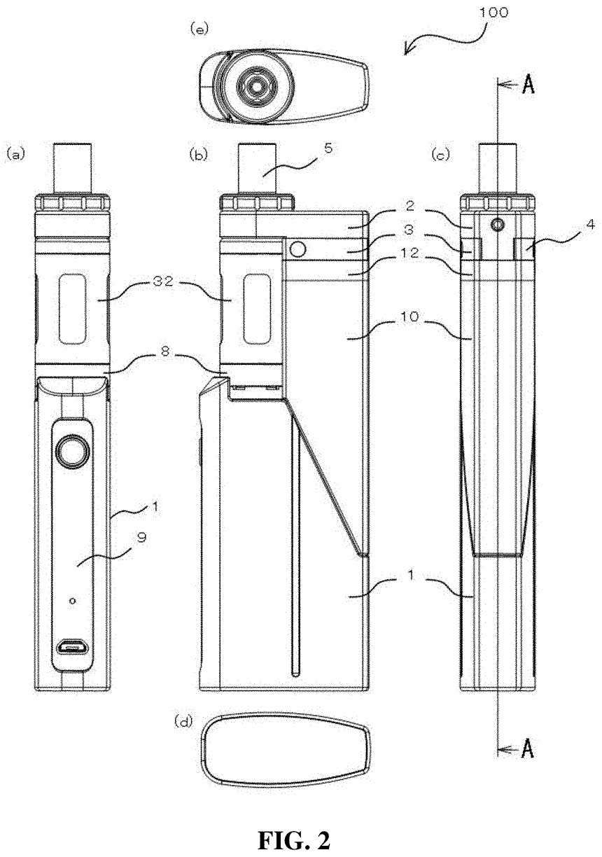

FIG. 1 is an exploded view exemplifying each member of the gas generator 100. Further, FIG. 2 shows a view from each direction of the gas generator 100 of FIG. 1, (a) is a left side view, (b) is a front view, (c) is a right side view, (d) is a bottom view, (e) shows a top view. In the present description, an up-and-down direction and a vertical direction mean an up-and-down direction on the page space and a vertical direction on the page space in FIG. 2(b), and a width direction, a lateral direction and a side portion side mean a right-and-left direction on the page space, a lateral direction on the page space and a right-and-left side portion side on the page space in FIG. 2(b).

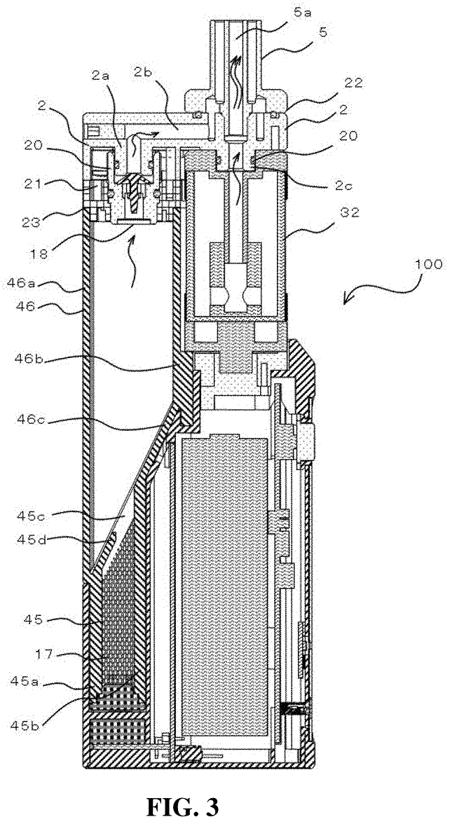

Further, FIG. 3 shows a cross-sectional view taken along line A-A of FIG. 2 (c) of the gas generator 100 of FIGS. 1-2.

Hereinafter, the gas generator 100 will be described mainly with reference to the assembled and exploded view of FIG. 1, and for convenience of description, other drawings will be referred to.

FIG. 1 as described above shows a configuration example of each member of the gas generator 100. A body cover 1 is a case made of a resin in which a battery receiving portion 43 opened upward and into which an entire battery 36 is inserted/stored in the vertical direction from the opening and an electrolysis tank receiving portion 44 having a shape arranged in parallel in the vertical direction with the battery receiving portion 43 and into which a reduced diameter portion 45 on a lower part of the electrolysis tank 10 can be inserted and fitted from above are provided. A battery 36 used here is preferably a charging-type lithium battery.

The main body cover 1 has a shape which is long in the side of the battery reception portion 43 and is cut in such a manner that an upper portion is inclined laterally in the side of the electrolysis tank reception portion 44. The base of battery 36 body cover 1, with the body bottom cover 6 as a closing member, opens and closes the base of battery acceptance 43, and closes the base of battery acceptance 43 with the body bottom cover 6 after inserting battery 36 from the bottom during assembly. The main body bottom cover 6 is closed by a cross recessed head screw 38. Further, the body cover 1 is provided with two control board (electronic board) 33, 42 so as to sandwich battery 36 in the longitudinal direction on both sides of battery receiving portion 43, the side control board 33 of the side surface of the body cover 1 is a main control board, the suction unit 32 (aromatic generator) and the mesh electrode 17 (electrode plate) to control the power supply from battery 36 to control board 42 of electrolysis tank 10 side for supplying power to.

A decorative laminated sheet 9 is attached to the side surface of the body cover 1 along the longitudinal side surface, and a button hole 9a through which an operation button 35 to the control substrate 33 is seen, a hole 9b for LED for light irradiation from an LED substrate 30, and a hole 9c for charging connector for connecting a connector for charging the battery 36 from an external power source are provided on the decorative laminated sheet 9 in this order from the top.

By pressing on the operation button 35 three times, a power supply signal is transmitted in the control substrate 33 to the control substrate 42, and power of the battery 36 is supplied for a predetermined time to a pair of the mesh electrodes (electrode plates) 17 through a housing 31 for substrate connector and a crimping substrate 28. When the power is supplied to the mesh electrode 17, the power supply signal is transmitted in the control substrate 33 to the LED substrate 30, and the LED substrate 30 causes the LED to emit light. As a result, the user can visually recognize that hydrogen or the oxygen-gas generating condition is established by 9b of the holes for LEDs. Incidentally, it was the condition of the power supply to the mesh electrode 17 to press the operation button 35 three times when the user moves by turning on the gas generator 100 in a pocket or the like, unintentionally button operated, it is a safety condition for avoiding that the power is supplied.

The two paired mesh electrodes 17 are arranged side by side longitudinally toward the above, respectively form positive and negative electrodes, and correspond to the electric power from the positive and negative electrodes of the battery 36. Moreover, an upper end of the mesh electrode 17 has a shape cut out diagonally so as to correspond to a boundary line between the reduced diameter portion 45 and a water storage body portion 46 of the electrolysis tank 10. A lower end of the mesh electrode 17 is raised up on a terminal substrate 28 and a rod-shaped titanium electrode 16 is coupled thereto in such a manner as to achieve an electrical connection. In order to shield the mesh substrate 17 and the terminal substrate 28 from water in a state in which the mesh electrode 17 is raised, there are provided a packing 13 (made of a resin such as silicone) which is installed on the terminal substrate 28, and an O-ring (made of a resin such as silicone, hereinafter refer also to as O-ring) which is attached to a periphery of the titanium electrode 16.

The electrolysis tank 10 is a container for storing water, the reduced diameter portion 45 and the water storage body portion 46 are integrally formed in order from below, and they are connected to each other therein fluidically. The water storage body portion 46 is opened upward so that water can be poured in and is half-closed by attaching an electrolysis tank lid 12. The electrolysis tank lid 12 passes through up and down, and is provided with a through opening 12a which receives the umbrella valve 23 and a screw cap 14. Water storage main body portion 46, the outer portion 46a forms a substantially flat side wall laterally over the lower end from the upper end as shown in FIG. 3 is connected as it is to the upper end of diameter-reduced portion 45, the inner portion 46b of the body cover 1 side from the upper end to the central lower position is formed parallel to the outer portion 46a, has a bottom 46c which is inclined bent from the central lower position. The bottom portion 46c extends to an intermediate position in a lateral direction and is coupled to the upper end of the diameter-reduced portion 45.

Further, diameter-reduced portion 45 is thinner than water storage main body portion 46 as described above, the upper end of the outer portion 46a of the side wall side as shown in FIG. 3 extends to the lower end continuously connected to the lower end of the outer portion 46a of water storage main body portion 46, the upper end of the inner portion 45b of the body cover 1 side bent downward at the position of the distal end (edge portion) of the bottom portion 46c of water storage main body portion 46 It extends to the lower end parallel to the inner portion 45b.

Furthermore, in the connecting position between the upper end of the outer portion 46a of the lower end and diameter-reduced portion 45 of the outer portion 46a of water storage main body portion 46, the water shielding plate 45d extending substantially the same inclined opening 45c and the bottom portion 46c of water storage main body portion 46 is provided. The impervious plate 45d extends over the interior of the paper surface vertical direction of the entire area of FIG. Therefore, even in the case that the aqueous solution accumulated in the electrolysis tank 10 is electrolyzed and the water storage amount is reduced, water is always accumulated approximately in the entire area of the inner portion of the diameter-reduced portion 45. Specifically, even when the water storage amount is reduced and the air layer is generated partly within the electrolysis tank 10, the diameter-reduced portion 45 is filled with water in the normal standing state and the air layer is not generated until water storage amount is reduced greatly since the diameter-reduced portion 45 is first of all thinner than the water storage main body portion 46.

Even if the water storage volume is reduced to some extent, an air layer may be generated in diameter-reduced portion 45 when the gas generator 100 is inclined or placed horizontally. However, in the case of the present electrolysis tank 10, water may be filled in diameter-reduced portion 45 even in such a case. Specifically, in the case of being inclined in the leftward direction on the page space of FIG. 3, the bottom portion 46c serves as a baffle plate and the air layer is formed in the inside portion 46b side of the water storage main body portion 46. Conversely, when inclined in the right direction of the paper surface of FIG. 3 water blocking plate 45d becomes a baffle plate air layer is formed only on the outer portion 46a side of water storage main body portion 46. Thus, the meshed electrodes 17 disposed in diameter-reduced portion 45 are constantly contacted entirely with water, so that hydrogen or oxygen-generating quantity can be constantly ensured even when the user inhales laterally.

An upper end edge of the mesh electrode 17 is formed by being cut out diagonally so that the electrode is soaked in water in the reduced diameter portion 45 without a gap by following the shapes of the reduced diameter portion 45 and the opening 45c. Returning to FIG. 1, the lower end of electrolysis tank 10 is closed by electrolysis tank bottom 11, but electrolysis tank bottom 11 is provided with a pair of through-holes into which the mesh electrodes 17 are inserted, and when diameter-reduced portion 45 of electrolysis tank 10 is inserted into electrolysis tank receiving portion 44 of the cover body 1, the mesh electrodes 17 pass through the through-holes of electrolysis tank bottom 11 and are positioned in diameter-reduced portion 45.

A description will be given of the umbrella valve 23 which is installed to the through opening 12a of the electrolysis tank lid 12 in the upper end of the electrolysis tank 10 When the screw cap 14 having an opening in its above and passing through up and down is installed to the through opening 12a, a vent filter 18 is interposed between a hole in a bottom portion of the screw cap 14 and a bottom portion of the through opening 12a, and an O-ring 21 is inserted into a downward periphery of the screw cap 14. The vent filter 18 is a micro hole and has a function of preventing water/dusts while adjusting an internal pressure in the opening of the screw cap 14. Moreover, the O-ring 21 shields a space between an outer peripheral wall of the opening in the screw cap 14 and an inner peripheral wall of the penetrating opening 12a from water.

Further, in the opening of the screw cap 14 amplifier valve 23 which operates in the vertical direction (made of a material having flexibility such as silicon) is attached, nozzle 5 (described later) user sucks the suction upward negative pressure acts amplifier valve 23 is raised operation, the through hole of the bottom of the screw cap 14, the through-hole 12a of electrolysis tank lid 12 It is fluidly connected to the inside of electrolysis tank 10 through. Therefore, when nozzle 5 is sucked in, hydrogen or oxygen gas rising and stored in electrolysis tank 10 is discharged to the outside. Conversely, when the user interrupts the suction and the negative pressure does not act, the ampliler condition 23 moves downward, the through hole in the bottom of the screw cap 14 is closed, and the discharge of hydrogen or oxygen gas in electrolysis tank 10 is closed.

To the electrolysis tank lid 12 to which the screw cap 14 and the umbrella valve 20 are attached, a mixer 2 is attached from above. Mixer 2 has a tubular member 2a extending downwardly as shown in FIG. 3, the tubular member 2a by inserting the lower end of the tubular member 2a into the opening of the screw cap 14 to form a flow path for guiding hydrogen or oxygen gas from the umbrella valve 23 upward. An O-ring 20 is provided around an outer peripheral wall of the tubular member 2a, and seals a clearance gap between the inner wall of the opening of the screw cap 14 and the tubular member 2a of the umbrella valve 20.

The fixing of the mixer 2 and electrolysis tank lid 12 is made by attaching the locking buttons 3 and 4. Each of the lock buttons 3 and 4 is pinched in a back and forth direction (a vertical direction on the page space of FIG. 3) at the clearance gap position in the up-and-down direction between the mixer 2 and the electrolysis tank lid 12 and snaps. Further, as shown in FIG. 3, the mixer 2 is provided in its upper portion with a flow channel 2b toward the nozzle 5 direction. The flow path 2b is connected to a flow path formed by the cylindrical member 2a, and guides hydrogen or oxygen gas as indicated by an arrow in FIG. 3.

A description will be given of the aromatic heater portion 32 which generates the aromatic air.

First, a contact terminal 37 of the battery 36 is inserted into the upper-end opening of the battery receiving portion 43 of the body cover 1. The contact terminal 37 is formed by connecting a bottom part of a large-diameter cylinder and an upper part of a small-diameter cylinder, the bottom part is inserted into the opening in the upper end of the battery receiving portion 43, and power from the battery 36 is supplied to the aromatic heater member 32. Contact terminal 37 is fastened from above to the joint 37 by a countersunk screw 38 with a cross. The joint 38 is formed in such a manner that the bottom portion of the small-diameter cylinder is connected to an approximately large-diameter discoid upper portion, and the upper portion of the contact terminal 37 and the bottom portion of the joint 38 are fitted in a nested manner.

The aromatic heater member 32 is mounted on the upper surface of the joint 8, and is pinched by the joint 8 and the mixer 2 and is fixed to the main body cover 1 when attaching the mixer 2 mentioned above. The aromatic heater member 32 is a general-purpose device, and when power is supplied, an air with aroma is generated therein and is emitted upward. Further, the mixer 2 is provided with a tubular member 2c which extends downward in parallel to the tubular member 2a mentioned above, and an upper end of the aromatic heater portion 32 is connected to the tubular member 2c. Therefore, the aromatic air discharged from the fragrance heater section 32 passes through the cylindrical member 2c as shown by the arrows in FIG. 3, merges with hydrogen or oxygen gas flowing through the flow path 2b via the cylindrical member 2a, flows into nozzle 5, and is discharged into the mouth of the user.

The nozzle 5 is structured such that the approximately large-diameter discoid member in the bottom portion is integrally connected to the tubular member in the upper portion, and the bottom portion is installed onto the opening in a top surface which is fluidly connected to the tubular member 2c of the heater portion 32 in the mixer 2. As a result, hydrogen or oxygen gas from the flow path 2b and/or aromatic air from the cylindrical member 2c are discharged from the inside of nozzle 5 to the outside of the upper end. An O-ring 22 is arranged in a connection portion between the bottom portion of the nozzle 5 and the mixer 2, and the connection portion is sealed.

Moreover, the aromatic heater member 32 controls power supply from the battery 36 by the control substrate 33. As described above, the power to the mesh electrodesubstrate 17 is supplied for the predetermined time by pressing on the button 35 attached to the body cover 1 three times. As mentioned above, the electric power to the mesh substrate 17 is supplied for a predetermined time by pushing the button 35 attached to the main body cover 1 three times. In the meanwhile, when the button is held down, the control board 33 connects the contact terminal 37 under a condition that the electric power supply signal is not transmitted to the mesh electrode 17, so that the electric power from the battery 36 is supplied to the aromatic heater portion 32 for a predetermined time.

Therefore, when the user inhales nozzle 5 by pressing the button 35 three times, hydrogen or oxygen gas is released from nozzle 5, and hydrogen or oxygen gas aspiration can be enjoyed for a predetermined period (while the LED board 30 emits light), and when the button 35 is pressed for a long time, aromatic hydrogen or oxygen gas can be enjoyed.

While embodiments of the gas generator used in the health care system of the present invention have been described above, it will be apparent that the gas generator is not limited to the embodiments described, but may be modified in design within the scope of common knowledge to those skilled in the art.

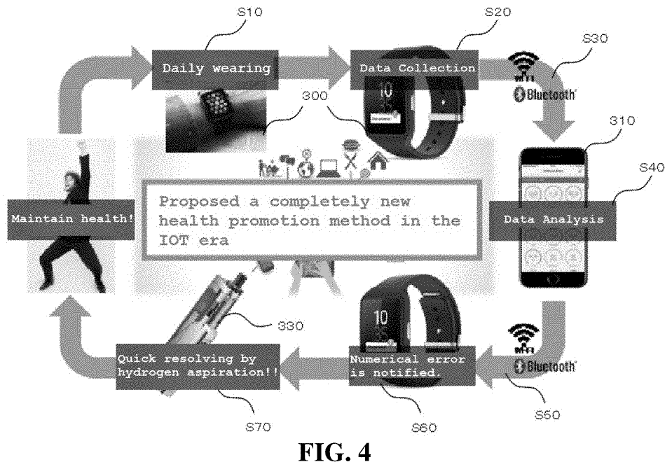

Next, referring to FIGS. 4 to 11, a health management system and a program of the present invention utilizing the above-mentioned gas generator will be described.

FIG. 4 shows an overview of the health management system of the present invention. In FIG. 4, the user first wears a wristwatch-type wearable portable terminal 300 in step S10. In part S20, the wearable portable terminal 300 digitizes the vital sign of the user from the contact portion and collects the digitized vital sign as data. The collected vital signs are transmitted to the mobile communication terminal 310 by an external radio transmitter such as Wifi, Bluetooth®, IR, zigbee, or the like (S30). The transmitted vital signs are analyzed by calculation means 320 (not shown) provided in the portable communication terminal 310 (S40). When the numerical value of the result of the analyzed vital sign is abnormal, the result is transmitted to the wearable portable terminal 300 by the external wireless transmission means in step S50. In step S60, the user confirms the abnormality of the transmitted numerical value from the wearable portable terminal 300. Then, the user aspirates the gas from the portable gas generator 330 (the above-mentioned hydrogen gas aspirator, corresponding to the gas generator 100) in view of the type, discharge quantity, and duration of the gas appropriate for the abnormality (S70). The S10-S70 cycle system allows users to easily and accurately manage their own health.

As another embodiment, in FIG. 4, a health system may be considered in which the wearable portable terminal 300 is provided with the calculation means 320 provided in the portable communication terminal 310. Specifically, the vital signs collected in S20 are analyzed by the calculation means 320 (not shown) provided in the wearable portable terminal 300 (S 40′). Then, the user confirms the abnormality of the numerical value in S60 from the wearable portable terminal 300.

Next, each step of S10 to S70 will be further described.

In S 10, the user wears the wearable portable terminal 300. Here, the wearable portable terminal 300 is worn by directly or almost directly touching the human body of the user, thereby obtaining the vital signs of the blood pressure, the pulse, the body temperature, and the like of the user. In FIG. 4, the patient is worn on his/her arm, but the setting for wearing the patient at another part where the vital signs can be appropriately acquired in the health care system can be selected.

In S 20, the vital signs are digitized and collected as data. The collected data includes, for example, blood pressure, pulse, body temperature, and the like, but information obtained when the wearable portable terminal 300 contacts the human body can be collected.

In S 30, vital signs are transmitted from the wearable portable terminal 300 to the portable communication terminal 310 by the external wireless transmission means. The transmission means is preferably wireless, although wired may be used if desired. In FIG. 4, Wifi or Bluetooth® is given as a specific example, but a common radio transmitting unit, for example, infra-red rays, zigbee rays, or the like, is employed.

In S 40 (or S 40′), the vital sign is analyzed (detailed later) by the calculation means 320 (not shown). The results analyzed may include abnormal values of vital signs, required gas and gas emissions and/or time appropriate for a user having the abnormal values, and the like.

The determination of whether or not the vital sign is abnormal may be performed by the wearable terminal after S20.

In S50, similarly to S30, by the external wireless transmission means, the mobile communication terminal 310 is transmitted to the wearable mobile terminal 300.

In S 50, abnormal information of the numerical value of the result of the analyzed vital sign, the necessary gas and gas discharge amount and/or time appropriate for the user, and the like are transmitted.

In S60, the analysis result is confirmed by the user on the display from the wearable portable terminal 300. The user can confirm abnormal values, health (values indicating health status), required gas and gas discharge amounts and/or time, etc.

In S 70, the user aspirates gas from the portable gas generator 330. The user performs gas suction of an appropriate gas type, discharge amount, and time based on the abnormal numerical values confirmed in S60, health (numerical values indicating a health state), required gas and gas discharge amount, and/or time, etc., thereby enabling health management suitable for the user.

In S70, the analysis result transmitted to the wearable portable terminal 300 may be transmitted to the portable gas generator 330 by the wireless transmission means, and the gas of an appropriate type, discharge amount, and time may be automatically discharged from the portable gas generator 330. Appropriate gas aspiration can be performed without the user making a self-judgment, and simpler and more precise health management is possible. In this case, it may be unnecessary to confirm the analysis result of the user in S60.

In addition, the type of gas to be sucked in S70 is not limited to hydrogen and oxygen, and a gas appropriate for healthcare can be adopted, and a plurality of gases and volatile components such as a flavor considering relaxation effects can be mixed.

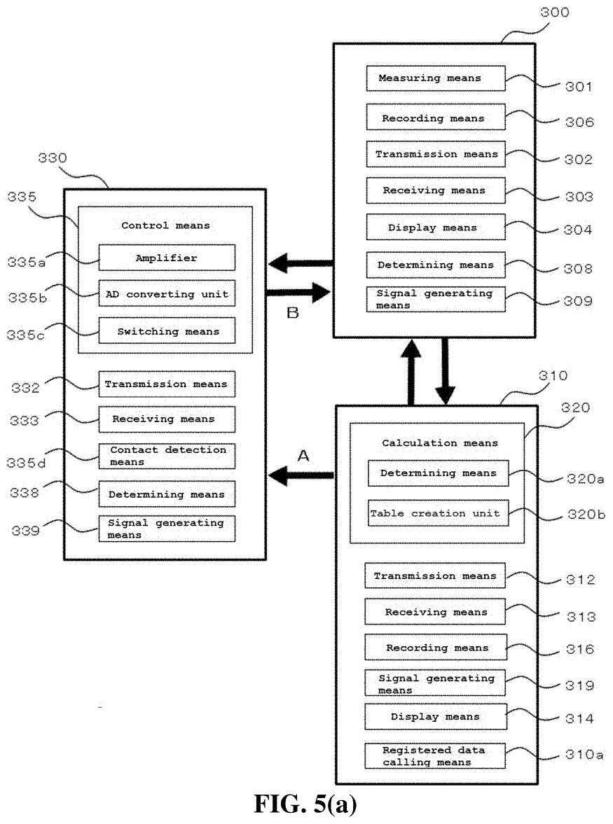

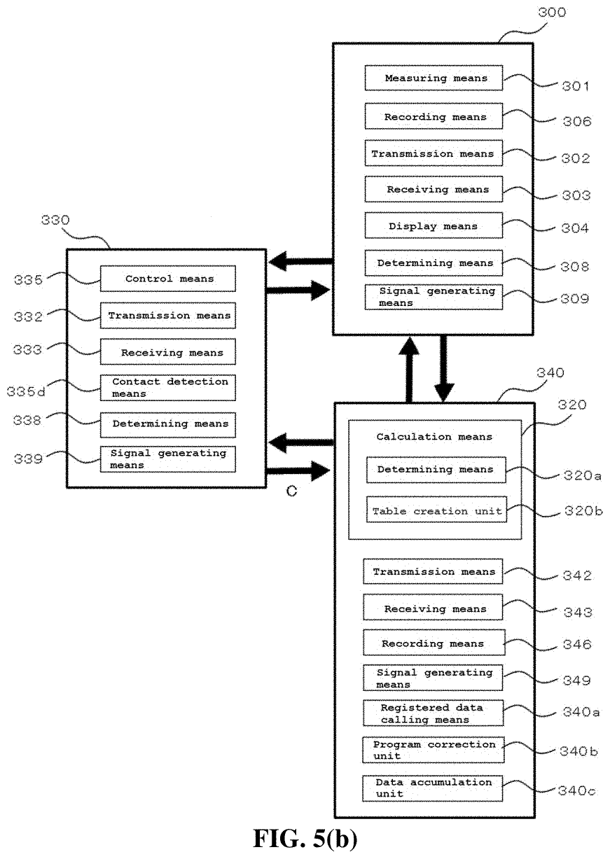

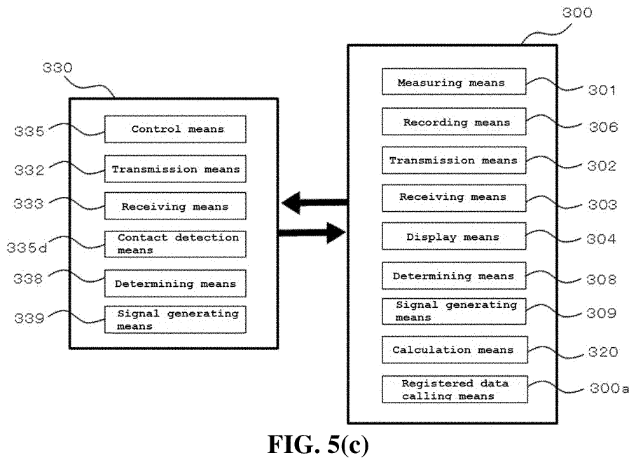

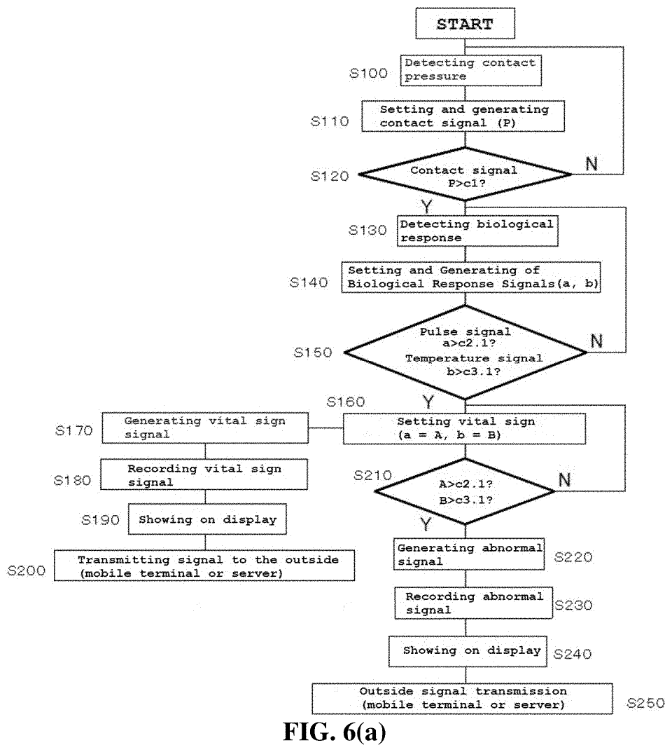

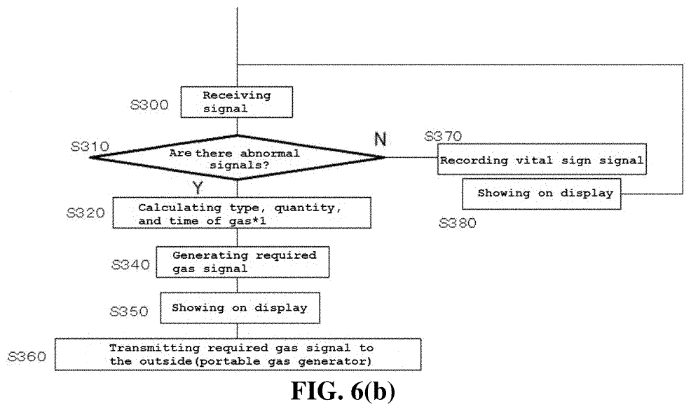

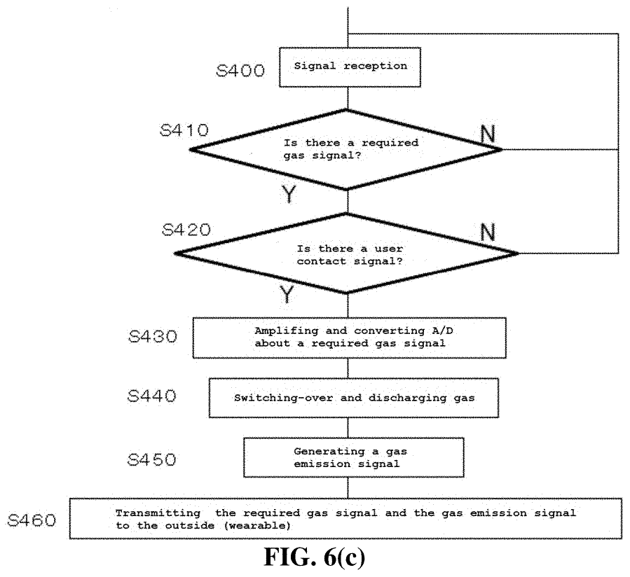

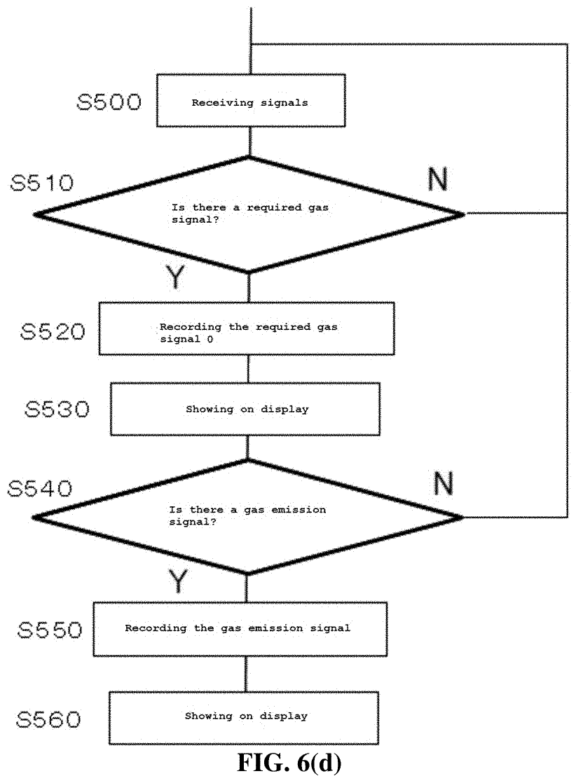

Next, an example of the program of the health management system will be described with reference to FIGS. 5 to 7. FIGS. 5(a)-(c) show the program for each different example of this health management system, and FIGS. 6(a)-(d) and 7(a)-(c) show the program flow.

First, the wearable portable terminal 300 will be described with reference to FIGS. 5A and 6A.

The wearable portable terminal 300 has generally measuring means 301, transmitting means 302, receiving means 303, display means 304, recording means 306, determining means 308, and signal generating means 309.

First, the measuring means 301 determines whether or not the user touches a predetermined part of the wearable portable terminal 300. The contact pressure to the predetermined portion is detected (S100), set and generated as the contact signal P (S110), and whether the contact signal P is larger than the predetermined value c1 is determined by the determining means 308 (hereinafter, “determination” is performed by the determining means 308; hereinafter, the description of “means” is omitted as appropriate) (S120). If P>c1, it is determined that the user is in contact. When it is determined that the user is touching, a biological reaction is detected (S130), and a biological reaction signal is generated and set (S140) by the signal generation unit 309. In the example of FIG. 6A, a biological reaction signal is formed by the pulse signal a and the body temperature signal b. Then, it is determined whether the pulse signal a and the body temperature signal b exceed the predetermined values c2.1 and c3.1, respectively (S150), and when they exceed, a=A and b=B are set as actual pulse and body temperature rather than noise (=vital signs) (S160).

Next, a signal of a set vital sign is generated (S170), recorded by recording means 306 (S180), and a vital sign signal is displayed (S190) by display means 304 and transmitted externally by transmitting means (S200). When the vital signs A and B thus set exceed the predetermined values c2.2 and c3.2 (S210), it is determined that the vital signs are abnormal, and abnormal signals are generated (S220), recorded (S230), displayed (S240), and transmitted (S250).

Next, the portable communication terminal 310 will be described with reference to FIGS. 5A and 6B.

The portable communication terminal 310 generally includes a transmitting means 312, a receiving means 313, a display means 314, a recording means 316, a signal generating means 319, a registered data calling means 310a, and a calculating means 320.

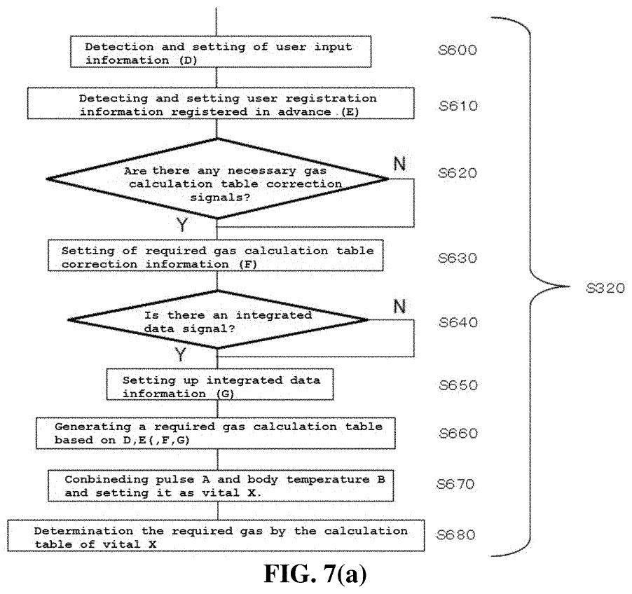

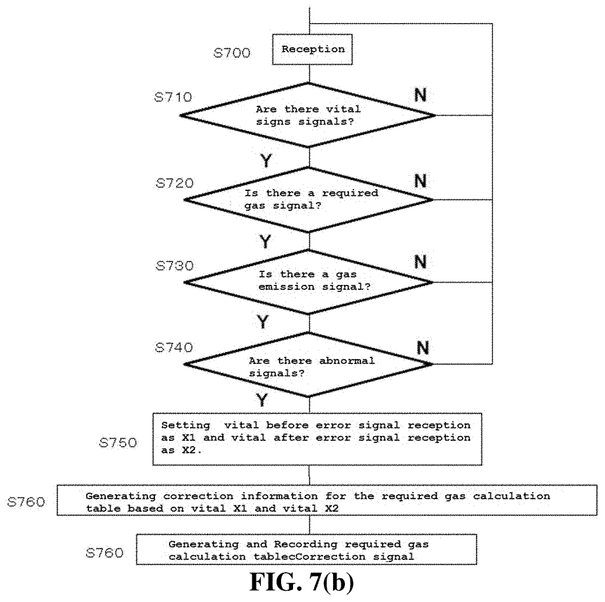

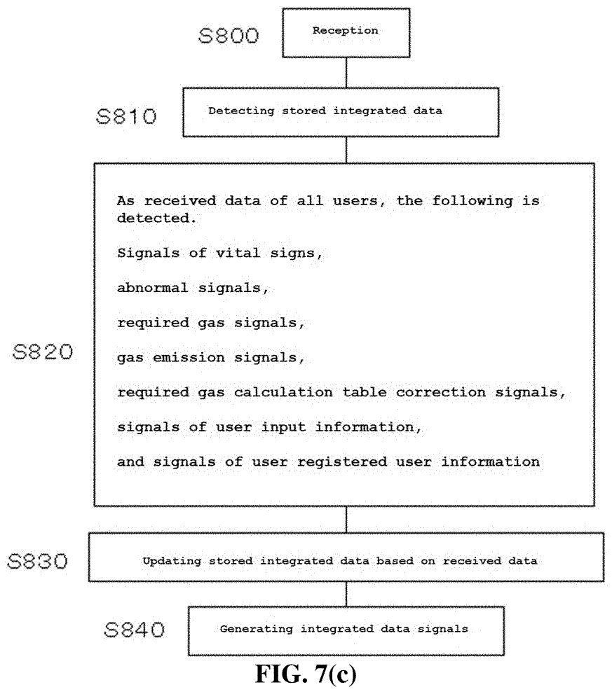

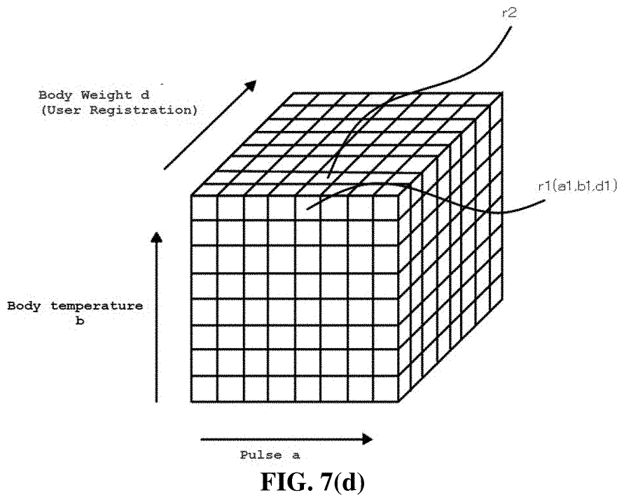

First, a signal transmitted from the wearable portable terminal 300 by the receiving means 313 is received (S300), it is determined whether there is an abnormal signal (S310). When there is an abnormal signal, calculating means 320 and the registered data calling means 310a are used to calculate the type, amount, and time of the gas based on the vital sign signal (details will be described later with reference to S320 and FIG. 7). When the type, quantity, and duration of the gas are calculated, required gas signals are generated (S340), displayed on a display (S350), and transmitted to the outside (portable gas generator 330) by the transmitter 312 (S360).

If there is no abnormal signal, the vital sign signal is recorded (S370) and the display is displayed (S380). Transmitting (S360,S380) destination of the vital sign signal is finally portable gas generator 330, when transmitting directly (arrow A in FIG. 5 (a)) and may be transmitted via the wearable portable terminal 300. In the case of passing through the wearable portable terminal 300 (arrow B in FIG. 5A), the display means 304 can display the necessary gas signal so that the user can confirm the necessary gas signal on the wearable portable terminal 300.

Next, FIG. 5 (a), a description will be given portable gas generator 330 with reference to FIG. 6 (c).

Portable gas generator 330 has generally transmitting means 332, receiving means 333, control means 335, contact detecting means 335d, determining means 338, a signal generating means 339.

First, signals transmitted directly or indirectly from the portable communication terminal 310 are received by the receiving unit 332 (S400). Then, in order to determine whether there are required gas signals (S410), and if so, whether the user is ready to use the portable gas generator 330, the contact detecting means 335d detects whether there is a contact at a predetermined portion and the determining means 338 determines (S420). Although S420 is a flow similar to the determination (S100 to 120) of the contact signal in the wearable portable terminal 300, it is not shown and described. If there is a contact signal, the required gas signal received is amplified by the amplifier 335a of the control unit 335, AD converted by the AD converting unit 335b (S430). Then, by switching the charge switch to the electrodes by the switching means 335c, to release the gas and its amount and time determined based on the required gas signal (S440). In addition, a gas emission signal is generated (S450) and a required gas signal and a gas emission signal are transmitted to a wearable portable terminal 300 (S460).

When there are no signals in the determination of S410 or S420, the gas is not released.

As an explanation of the embodiment of FIG. 5A, a flow after the wearable portable terminal 300 receives a signal transmitted from the portable communication terminal 310 will be described with reference to FIG. 5A and FIG. 6D.

First, a signal is received (S500) and it is determined whether or not a required gas signal is present (S510). If there are signals, the gas, quantity, and duration required for the user are recorded (S520) and displayed (S530). Then, it is determined whether there is a gas emission signal (S540), if there is a signal, records that the gas has been released (S550), and displays it (S560).

In the absence of a signal, no recording or display is performed.

In the embodiment of FIG. 6, only the pulse and the body temperature are used as vital signs (biological reactions) measured by the wearable portable terminal 300, but other information such as blood pressure, exercise information, and calories consumed (for example, the number of steps per hour is recorded) can be used as vital signs. Further, in the step of judging the abnormality of S210 or the like, a predetermined criterion is defined and set, but, for example, the step of judging the abnormality when the blood pressure is out of a predetermined range is adopted. Whether or not the state of the user is abnormal (i.e., the abnormal signal should be generated) is judged not only by individual vital signs but also comprehensively.

The program flow in the order of (a) to (d) of FIG. 6 has been described above in the embodiment of FIG. 5A.

Next, with reference to FIGS. 7(a) and 5(a), a description will be given of a calculation (S320) of the type, amount, and time of gas based on vital sign signals by the calculation means 320 of FIG. 6(b).

First, information which cannot be measured and often changed by the wearable portable terminal 300 such as the user's weight and food volume, entered by the user is detected and set by the registered data calling means 310a (S600). Similarly, information which cannot be measured and generally variable by the wearable portable terminal 300 such as the user's gender, such as that input by the user is detected and set (S610). Next, it is determined whether or not a required gas calculation table correction signal (described later in FIG. 7(b)) or an integrated data signal (described later in FIG. 7(c)) is received, and if the correction signal is received, the signal information is set as signal information (S620,S630 and S640,S650). Then, the table creation unit 320b generates a necessary gas calculation table based on the set data (S660). The necessary gas calculation table is a program set to calculate necessary gas, amount, and time suitable for the vital sign at that time for each input/registration information of the user. The vital sign of the user at that time is set as vital X (S670), and a search (calculation) is performed in the required gas calculation table to determine the required gas, quantity, and duration (S680).

For example, as shown in FIG. 7D, gas information corresponding to a “room” based on setting information such as vital signs is set as a necessary gas. When the pulse, the body temperature, and the body weight are a1, b1, and d1, respectively, the required gas is set corresponding to the “room” of r1. In FIG. 7(d), it is a “room” table in which only three setting information are selected, but actually, the “room” is selected by referring to other setting information.

In order to explain the necessary gas calculation table correction signal and the integrated data signal, the description will be made with reference to FIG. 5(b) which is another embodiment of FIG. 5(a) together with FIGS. 7(b) and 7 (c). FIG. 5B is a diagram in which a server 340 is employed instead of the portable communication terminal 310 of FIG. 5A, and a point different from that of FIG. 5A will be described below.

Unlike the portable communication terminal 310, the server 340 includes a program correction unit 340b and a data accumulating unit 340c. Further, from the portable gas generator 330, the server 340 gas emission signal (signal that emits gas) is transmitted (arrow C in FIG. 5 (b)).

The program correction unit 340b generates and transmits the necessary gas calculation table correction signal. As shown in FIG. 7(b), first, in the signal received by the server 340, the presence or absence of a vital sign signal, a required gas signal, a gas emission signal, and an abnormal signal is determined (S710˜S740). When there are all signals, there is an abnormality in the user's condition, and the required gas is calculated, and it is judged that there is still an abnormality in spite of the user's suction of the necessary gas, and the correction of the necessary gas calculation table is performed. Specifically, the vital signs before and after the abnormal signal reception is set (S750), based on the difference or the like, for example, to generate correction information of the required gas calculation table such that more gas is required even in the same condition (S760). Then, it is signalized (S770).

In FIG. 5B, although the calculation means 320 is provided in the server 340, when the calculation means is provided outside the server, the necessary gas calculation table correction signal is transmitted to a portable terminal such as a smart phone having the calculation means.

The correction will be described schematically. For example, as shown in FIG. 7D, first, the room r1 is adopted when the pulse, the body temperature, and the body weight are (a1, b1, d1), but when the abnormality of the vital sign continues even if the gas is sucked, the correction is carried out on the basis of the abnormality of the vital sign, and even when the necessary gas information of the room r2 is adopted even if the abnormality of the vital sign is (a1, b1, d1) from the next time.

The data accumulating means 340c receives information of all users and collects the information in a cloud form, and generates an integrated data signal. As shown in FIG. 7(c), first, the signal is received (S800). Then, the stored integrated data is detected (S810), and received signals of all users are detected (S820). The received signal relates to the present health management system, such as vital sign signal, abnormal signal, required gas signal, gas emission signal, required gas calculation table correction signal, signal of user input information, signal of pre-registered user registration information, required gas calculation table correction signal, etc. Then, based on the received signals, the stored integrated data is updated (S830) and the new integrated data is signalized (S840). Aggregated data is updated every time it is used by users worldwide.

As in the case of the required gas calculation table correction signal, although FIG. 5(b) has a calculation unit 320 in the server 340, when there is a calculation unit outside the server, transmits an integrated data signal to a portable terminal such as a smartphone having a calculation unit, for example.

In the calculation (S320) of the type, amount, and time of gas based on the vital sign signal by the calculation unit 320, it is possible to set the required gas calculation table suitable for one user according to the use of one user and the whole world, such as the required gas calculation table correction signal or the integrated data signal described above. In addition, generation (S660) of a required gas calculation table using integrated data signals is optimized and speeded up by performing deep learning by AI.

FIG. 5(c) shows another embodiment of FIGS. 5(a) and 5 (b). In FIG. 5C, the wearable portable terminal 300 includes calculation means.

As a program flow, generally FIG. 6 (a), (b), (d) flow is performed in the wearable portable terminal 300, the flow of FIG. 6 (c) is performed in the portable gas generator 330.

In the case of FIG. 5 (c), the flow of the required gas calculation table correction signal (FIG. 7 (b)) and the integrated data signal (FIG. 7 (c)) in the calculation (S320) of the type, amount, and time of the gas, it may be employed to perform at the server 340 and transmit the signal to the wearable portable terminal 300.

Although the health management system and the programs of the present invention have been described above, as another embodiment, for example, a configuration in which the external destination of the required gas signals in S360 of FIG. 6B is the wearable portable terminal 300 instead of the portable gas generator 330, and the user looks at the wearable portable terminal 300 to adjust the gas emission of the portable gas generator 330 by the user himself/herself can be cited.

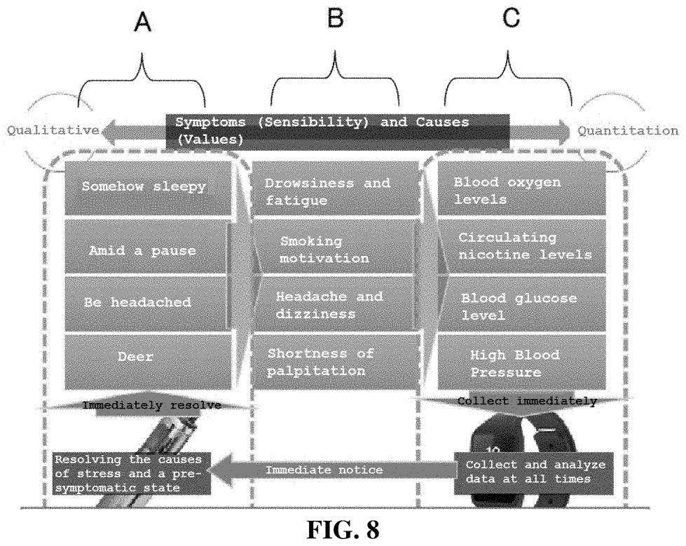

Embodiments of the use of the health care system will now be described with reference to FIGS. 8-11.

FIG. 8 shows an example of the effect of using a health management system utilizing a gas generator.

When the user feels qualitative sensation (A) such as “something sleepy” or the like, “sleepiness or fatigue” is generally the cause (B). At this time, if the wearable portable terminal 300 of the present health management system is mounted, the abnormality of the “blood oxygen concentration” (C) corresponding to the qualitative sensation and cause can be quantitatively collected as data. Then, appropriate gas can be suctioned based on the collected data, and the abnormality of (C) is resolved, the cause (B) is removed, and the abnormality of the sensation (A) felt by the user is also resolved.

That is, the present health management system can quantitatively analyze the qualitative sensation felt by the user and appropriately deal with it, and therefore has the effect of being able to easily and accurately deal with an abnormality that the user does not sense, rather than a coping therapy that relies on the user's own sensation.

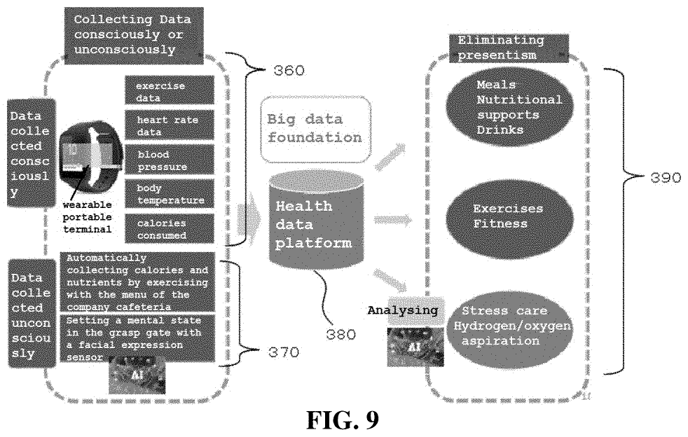

FIG. 9 shows an example of the use of the health care system.

Using this health care system, it is possible to construct a “health data platform” 380 of the user by accumulating data 360 (exercise data, heart rate data, blood pressure, body temperature, calories consumed in FIG. 6) consciously collected by the wearable portable terminal 300 of the health care system and data 370 (acquired calories, nutrients, and mental states automatically acquired in the company in FIG. 6) unconsciously collected by the wearable portable terminal 300 of the health care system into big data.

Various health management techniques 390 (e.g., meals, exercises, etc., hydrogen/oxygen suction in FIG. 6) are proposed on the platforms constructed, and the health management systems can be analyzed, etc., and appropriate hydrogen/oxygen aspiration 390a can be proposed.

By using this health management system, a platform for proposing various health management methods including hydrogen and oxygen suction can be constructed, and therefore, even health management by meals, exercises, and the like can be performed more easily and accurately than in the past without bothering the user by analyzing the platform data.

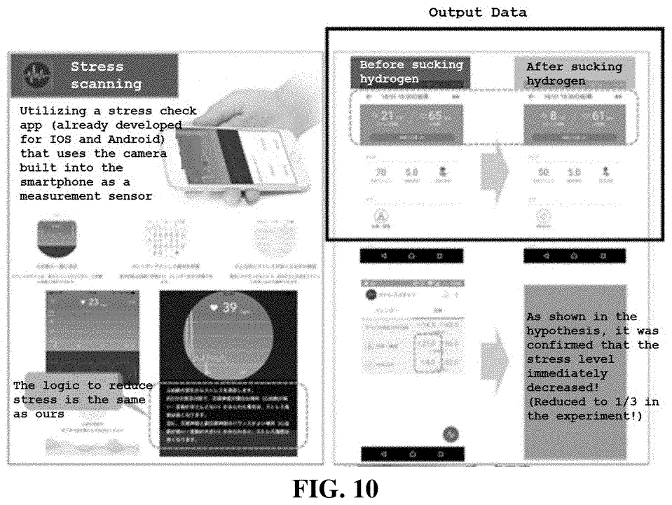

FIG. 10 illustrates another embodiment of the health care system.

It is also conceivable that the portable communication terminal 310 is provided with calculation means capable of comparing and outputting the user's past vital signs with the current vital signs. The present health care system makes it possible to immediately visually recognize the change in vital signs caused by performing gas suction, and the effect of the present health care system can be realized, which also leads to an improvement in the motivation of health care.

In FIG. 10, as an example, a calculation means that enables stress scanning using a camera is employed, and it is shown that the stress became one-third before and after gas suction. Although the stress state is output in FIG. 7, the data that can be output is arbitrary and may be appropriately selectable by the user.

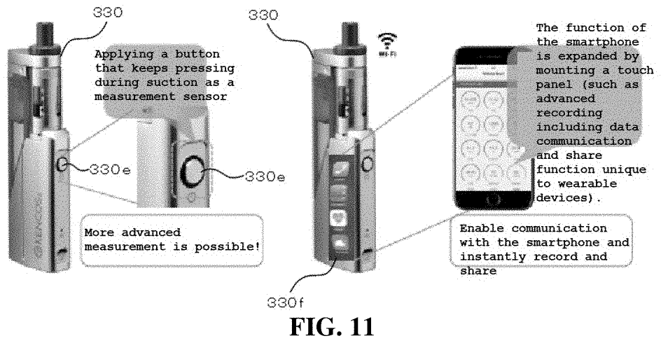

FIGS. 11A and 11B show an example in which the portable gas generator 330 itself is used as the wearable portable terminal 300 as another example of the health management system.

In FIG. 11 (a), the function as a sensor is also employed in the suction button 330e of the portable gas generator 330, to collect vital signs from the fingertips touching the sensor. The collected vital signs are transmitted to the portable communication terminal 310 by an external wireless transmission means and the analysis results are transmitted to the portable gas generator 330 after analysis (when the calculation unit 320 for analyzing is provided in the portable gas generator 330 it may not be transmitted).

Further, in FIG. 11 (b), and a touch panel 330f to the portable gas generator 330, by the touch panel operation, and data transmission to the portable communication terminal 310, the display of the analysis result, it is possible to perform suction operation or the like sensuously. By employing the touch panel, the convenience of the user can be improved.

In the health management system shown in FIGS. 11A and 11B, since the wearable portable terminal 300 and the portable gas generator 330 can be used in one article, the convenience of use and transportation of the user of health management in gas aspiration is greatly improved, and the motivation of the user for health management is also improved.

While embodiments of the health care system and program of the present invention have been described above, it will be apparent that the health care system and program are not limited to the embodiments described, but may be modified in design within the general scope of those skilled in the art.

Next, the results of examining changes in neural activity and/or circulatory activity of a user when hydrogen is ingested using a gas-generator used in the present healthcare system will be described.

The following validation tests were conducted to confirm that parasympathetic nerves are dominant and fatigue is reduced by oral aspiration of hydrogen, and also to confirm the duration of time until the physiological index changes.

In a validation test using a gas-generator, hydrogen produced by a gas-generator (electrolysis-type hydrogen gas suction tool 100 described later) is generally sucked orally. Specifically, hydrogen generated by hydrogen generator device 100 is aspirated in spontaneous respiration for about 10 minutes. Since 8 cc of hydrogen is generated per minute by the electrolysis method (at the same time, 4 cc of oxygen is generated), 12 cc of mixed gas of oxygen and hydrogen is generated per minute in the amount of hydrogen generated per 11 minutes. The gas mixture is sucked under natural breathing. Normally, in spontaneous breathing, the exhaled air contains up to 0.24% (0.18% hydrogen and 0.06% O2) of the gas mixture generated by aspirating about 5 liters of air per minute in an adult.

The gas generated from the gas generator (hereinafter referred to as hydrogen generator 100) is hydrogen and oxygen, and both hydrogen concentration and the oxygen concentration increase in the mixed gas from the atmosphere, but the respective concentration increases are hydrogen 0.18% and oxygen 0.06% as described above, while the respective concentrations in the atmosphere are hydrogen 0.5×10−4% (=0.5 ppm) and oxygen about 21%. Therefore, it can be considered that the oxygen concentration in the mixed gas hardly increases, and only hydrogen concentration increases.

Twenty subjects were selected and were assigned to two groups according to age and study time (morning and afternoon) and measured by 10 subjects per day. The selected subjects were healthy women in their 20 s to 30 s and excluded the following.

(1) Patients who smoke (2) are cold (including those who feel cold in the summer), (3) are currently undergoing drug treatment for any disease, (4) have been taking drugs or applying drugs (excluding common cold, including a history of treatment for pollinosis), (5) have a history of serious disorders such as liver, kidney, heart, lung, blood, etc., (6) have hypertensive symptoms such as systolic blood pressure of 160 mmHg or diastolic blood pressure of 100 mmHg or more in the past month, (7) have donated more than 200 mL or more within three months, (8) have undergone pregnancy, lactation, or pregnancy disease, (10) have undergone surgery within the past month, (9) have had a history of severe disorders such as cutaneous disease, etc. Those who have participated in other human clinical studies, and those who have not participated in other human clinical studies for one month (12), Those who are considered inappropriate for this study by the investigator.

In addition, subjects were required to take cautionary notes at the time of participation in the study: (1) sleep adequately (about 7 hours) on the previous day; (2) do not use irritants such as curry and kimchi, or caffeine beverages such as coffee and tea on the meal before the study; (3) do not use perfume or puffeume on the day of measurement; (4) on the day of measurement, the subject can accept the cosmetic product to be removed and measured by pigmentation; and (5) on the day of the study (good before the study), the test was performed by removing glasses and contact lenses.

The testing method is as follows:

(1) In this study, subjects should aspirate hydrogen.
(2) The physiological effects of hydrogen will be verified by testing and evaluating each subject as described below while aspirating hydrogen, and comparing and examining each evaluation before and after aspiration.
(3) For subjects who are sitting in a chair for measurement and are open, the study director or the study collaborator prepares a hydrogen generator (which does not generate hydrogen) as a control, and assigns them to the subject. The subject should attach a mouth to the nozzles 5 (see FIGS. 1 to 3) of electrolysis-type hydrogen gas suction tool 100 or the tubing connected to the nozzles 5 (see FIGS. 1 to 3) and breathe normally for about 10 minutes. Thereafter, the measurement shown in is performed at any time. Thereafter, the nozzles 5 of electrolysis-type hydrogen gas suction tool 100 for generating hydrogen as test samples or the tubes connected thereto are put in the mouth, and the hydrogen is sucked for 10 minutes in the same manner. Thereafter, each measurement is performed. The study director or study collaborator shall immediately discontinue aspiration if any abnormalities are observed in the subject while the subject is aspirating hydrogen. When hydrogen aspiration is discontinued, the practitioner shall record the discontinuation and the aspiration time on the Case Report Form.
(4) The study director shall confirm that no adverse events have occurred in the subject during the study period.

In this verification test of the bioactivation method for enhancing a neural activity and/or a blood circulation activity of a living body, specifically, the following tests were conducted and evaluated:

### 1) Measurement of Autonomous Nervous System

For the analysis of the action of the autonomous nervous system, the pupil-to-light reaction measurement method and the skin temperature measurement method of the palm part (index finger) which can measure in a short time with little burden of the examinee, though it is high sensitivity, are carried out. Their measuring methods are explained below.

1-1) Pupil Reflection to Light

After fitting a goggle measuring device for measuring the pupil diameter, the pupil was accustomed to night vision (usually in night vision for 2 minutes). By short-time irradiating the pupil with a very weak red light emitting diode light for about 0.2 seconds to 1.0 seconds, the pupil was temporarily constricted by the light reflection, and the pupil expanded quickly thereafter. Accordingly, the pupil diameter changes during the pupil constriction and dilation reaction during the period before and after the state are photographed with a highly sensitive CCD camera (instrument: iriscoder, Hamamatsu Photonics), and the changes in pupil diameter, pupil constriction speed, and pupil dilation speed are analyzed to determine whether autonomous nervous activity dominates sympathetic activity, or whether parasympathetic activity dominates. When the parasympathetic activity is dominant, the parasympathetic activity becomes dominant as the pupil diameter becomes smaller when the light is sensed, and as the pupil constriction rate (CR) increases, the parasympathetic activity becomes dominant as the pupil constriction rate (CR) increases.

1-2) Fingertip Temperature

Focusing on the physiological response that the skin temperature in the peripheral region (this time, the central forehead and the ventral part of the first joint of the index finger) changes due to superiority or inferiority of sympathetic nerve activity, the change in the skin temperature before and after drinking hydrogen water will be measured with temperature sensors over time. The temperature sensor body has a thickness of about 1 mm and a diameter of about 3 mm, and changes in skin temperature are measured from the sensor to a recorder by wire.

### 2) Measurement of Central Nervous System

Concerning the effect of central nervous activity by hydrogen, the activity (fatigue rate) of the brain is measured by the flicker device, while the brain stress test utilized for the purpose of evaluating the activity and stress of the brain is carried out. In addition, the effects of visual field function, skin sensation function, and center of gravity balance function, etc. are measured by the cerebral execution function meter. In addition, regarding mood emotion change, focus, sleepiness, etc. are interrogated with a multifaceted emotional state scale. The respective measurements will be described in brief below.

2-1) Brain Stress Test

Evaluate and analyze cerebral stress and cerebral turnover (activity) by sequentially touching the single-digit-single-digit (→2→per→, up to →3→ . . . → and →20) displayed on the monitor screen. This measurement is made immediately after the control and the subject sample (hydrogen) are suctioned.

2-2) Flicker Measurement

By judging the flashing frequency (flicker value) of the green LED light, whose frequency varies from 70 to 30 Hz, the activity of the brain (which can also be regarded as fatigue level) is measured. Specifically, it determines the frequency when the green feels flashing by continuously decreasing the frequency from 70 Hz. This measurement is repeated five times. This measurement is made immediately after the control and the subject sample (hydrogen) are suctioned.

2-3) Analysis (Measuring Instrument; Brain Execution Function Total, Manufactured by Anima Co., Ltd.) of the Effect on Brain Execution Function (Comprehensively Analyzing Left-Right Cognitive, Visual Field Function, Short-Term Memory, Skin Sensation, Barycentric Balance, Etc.).

Specifically, it is judged whether the white circle appearing on the personal computer is on the left or right side from the center line, and it is judged as soon as possible whether the button is pressed or whether the left or right vibration plate is vibrated, and it is judged as soon as possible that the button is pressed, and the moving distance of the center of gravity of the standing position is measured for 30 seconds on the center of gravity swing meter. This measurement is performed immediately after aspiration of the controls and test samples (hydrogen).

2-4) Multilateral Emotional State Scale

Twenty items of four subscales of “depression and anxiety,” “boredom,” “active pleasure,” and “inactive pleasure” were used. Subjective evaluation is performed immediately after aspiration of controls and test samples (hydrogen) with a five-point evaluation from “not at all sensed=0” to “clearly sensed=4”.

The aforementioned test results will be described.

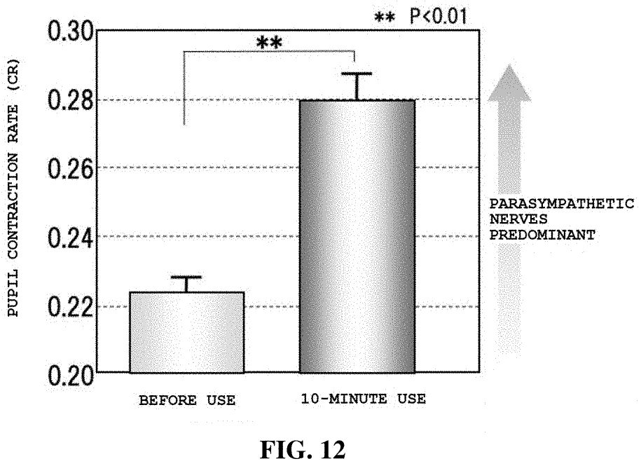

The result of the pupil reflection to light in 1-1) is as in the following Table 1, and FIG. 12 shows a measurement graph illustrating an average pupil contraction rate (CR value) of right and left eyes of the 17 subjects excluding those inappropriate from the subjects before and after suctioning of hydrogen. It was confirmed from this Table 1 and FIG. 12 that the pupil contraction rate (CR value) was significantly increased by hydrogen suctioning, and the parasympathetic nervous activity became predominant. This result can be considered to suggest a sedative effect by the hydrogen suctioning.

TABLE 1

| | CR value (right eye) | | | CR value (left eye) | | |
|---|---|---|---|---|---|---|
| Subject | Before | After | Difference | Before | After | Difference |
| 1 | 0.20 | 0.21 | +0.01 | 0.27 | 0.29 | +0.02 |
| 2 | 0.16 | 0.20 | +0.04 | 0.16 | 0.16 | ±0.00 |
| 3 | 0.03 | 0.03 | ±0.00 | 0.07 | 0.08 | +0.01 |
| 4 | 0.22 | 0.30 | +0.08 | 0.24 | 0.32 | +0.08 |
| 5 | 0.24 | 0.23 | −0.01 | 0.24 | 0.22 | −0.02 |
| 6 | 0.24 | 0.41 | +0.17 | 0.24 | 0.38 | +0.14 |
| 7 | 0.36 | 0.38 | +0.02 | 0.34 | 0.38 | +0.04 |
| 9 | 0.22 | 0.30 | +0.08 | 0.20 | 0.32 | +0.12 |
| 11 | 0.13 | 0.18 | +0.05 | 0.13 | 0.18 | +0.05 |
| 12 | 0.23 | 0.30 | +0.07 | 0.25 | 0.30 | +0.05 |
| 13 | 0.39 | 0.41 | +0.02 | 0.39 | 0.41 | +0.02 |
| 14 | 0.19 | 0.45 | +0.26 | 0.21 | 0.30 | +0.09 |
| 16 | 0.16 | 0.24 | +0.08 | 0.17 | 0.21 | +0.04 |
| 17 | 0.13 | 0.28 | +0.15 | 0.14 | 0.29 | +0.15 |
| 18 | 0.50 | 0.36 | −0.14 | 0.42 | 0.34 | −0.08 |
| 20 | 0.21 | 0.18 | −0.03 | 0.19 | 0.17 | −0.02 |
| 21 | 0.40 | 0.30 | −0.10 | 0.29 | 0.29 | ±0.00 |
| Ave. | 0.23 | 0.28 | +0.04 | 0.23 | 0.27 | +0.04 |

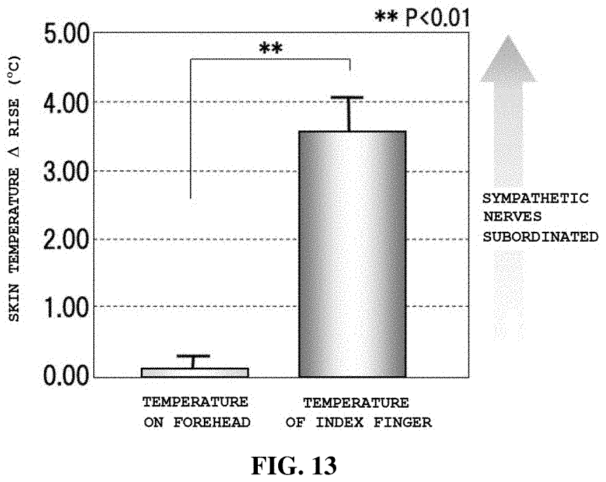

The results of 1-2) point temperature are shown in Table 2 below. As in FIG. 12, FIG. 13 shows a measurement graph showing the temperature of increase in the derma of the forehead (° C.) separately measured before and after the test for 17 subjects, before and after hydrogen inhalation, as shown in FIG. 12. It was suggested from this Table 2 and FIG. 13 that peripheral skin temperature was significantly raised by hydrogen suctioning, the sympathetic nervous activity was suppressed, and the parasympathetic nervous activity became predominant.

TABLE 2

| | Fingertip temperature | | |
|---|---|---|---|
| Subject | Before | After | Difference |
| 1 | 27.20 | 30.85 | +3.65 |
| 2 | 32.29 | 31.55 | −0.74 |
| 3 | 30.48 | 32.82 | +2.34 |
| 4 | 27.50 | 31.93 | +4.43 |
| 5 | 27.79 | 30.99 | +3.20 |
| 6 | 24.30 | 31.28 | +6.98 |
| 7 | 26.55 | 31.14 | +4.59 |
| 9 | 31.28 | 32.52 | +1.24 |
| 11 | 24.50 | 31.67 | +7.17 |
| 12 | 29.97 | 32.57 | +2.60 |
| 13 | 24.87 | 31.73 | +6.86 |
| 14 | 29.06 | 32.33 | +3.27 |
| 16 | 23.55 | 31.03 | +7.48 |
| 17 | 32.53 | 32.13 | −0.40 |
| 18 | 24.53 | 31.59 | +7.06 |
| 20 | 30.75 | 31.93 | +1.18 |
| 21 | 31.84 | 32.04 | +0.20 |
| Ave. | 28.18 | 31.77 | +3.59 |

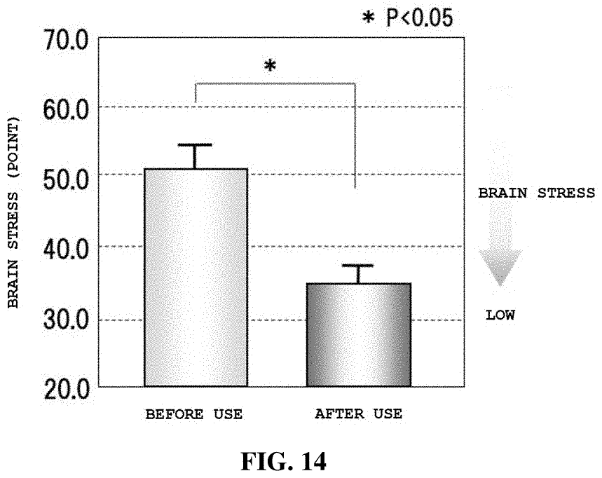

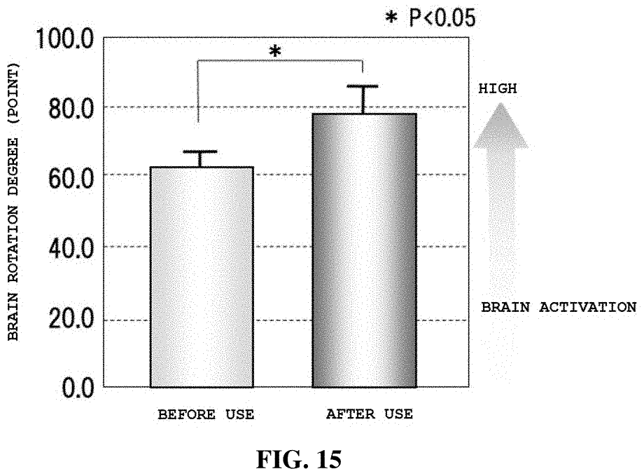

The results of 2-1) brain stress test and 2-2) flicker measurements (including brain age evaluation) are shown in Table 3 below, and FIG. 14 is a graph showing the mean of 17 subjects before and after hydrogen aspiration for the brain stress evaluation scored from Table 3, similarly to FIGS. 12 and 132. It was suggested from this Table 3 and FIG. 14 that the brain stress was significantly decreased by the hydrogen suctioning and the stress reduction effect. FIG. 15 is a graph showing the mean of 17 subjects before and after hydrogen aspiration for the cerebral rotations evaluated by scoring from Table 3, similarly to FIGS. 12 to 14. It was suggested from this Table 3 and FIG. 15 that the brain rotation degree score was significantly raised by the hydrogen suctioning, and the brain function was activated.

TABLE 3

| | Flicker value (average) | | | Brain age (years old) | | | Brain stress (%) | | |
|---|---|---|---|---|---|---|---|---|---|
| Subject | Before | After | Difference | Before | After | Difference | Before | After | Difference |
| 1 | 44.7 | 45.3 | +0.7 | 22 | 20 | −2.0 | 30 | 68 | +38.0 |
| 2 | 41.7 | 41.3 | −0.3 | 46 | 44 | −2.0 | 51 | 26 | −25.0 |
| 3 | 35.7 | 35.0 | −0.7 | 29 | 30 | +1.0 | 39 | 11 | −28.0 |
| 4 | 47.3 | 51.3 | +4.0 | 19 | 31 | +12.0 | | 66 | +66.0 |
| 5 | 46.3 | 50.0 | +3.7 | 16 | 16 | ±0.0 | 58 | 56 | −2.0 |
| 6 | 41.0 | 41.3 | +0.3 | 26 | 23 | −3.0 | 45 | 97 | +52.0 |
| 7 | 49.0 | 50.0 | +1.0 | 25 | 24 | −1.0 | 79 | 27 | −52.0 |
| 9 | 46.0 | 49.7 | +3.7 | 17 | 16 | −1.0 | 38 | 37 | −1.0 |
| 11 | 43.0 | 43.0 | ±0.0 | 21 | 34 | +13.0 | 49 | 37 | −12.0 |
| 12 | 41.7 | 40.0 | −1.7 | 28 | 29 | +1.0 | 37 | 23 | −14.0 |
| 13 | 47.0 | 45.0 | −2.0 | 45 | 32 | −13.0 | 31 | 22 | −9.0 |
| 14 | 39.7 | 46.7 | +7.0 | 28 | 26 | −2.0 | 9 | 15 | +6.0 |
| 16 | 45.33 | 41.7 | −3.7 | 43 | 25 | −18.0 | 58 | 28 | −30.0 |
| 17 | 42 | 39.7 | −2.3 | 34 | 29 | −5.0 | 37 | 38 | +1.0 |
| 18 | 38.3 | 38.3 | ±0.0 | 32 | 32 | ±0.0 | 14 | 37 | +23.0 |
| 20 | 45 | 45.7 | +0.7 | 29 | 26 | −3.0 | 58 | 94 | +36.0 |
| 21 | 39.3 | 39.7 | +0.3 | 18 | 29 | +11.0 | 44 | 16 | −28.0 |
| Ave. | 43.4 | 43.75 | +0.6 | 28.1 | 27.4 | −0.7 | 42.3 | 41.1 | +1.2 |

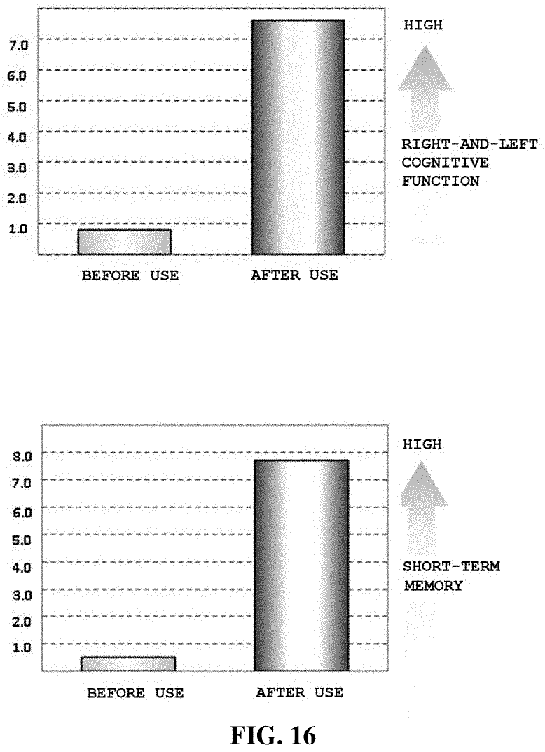

The result of 2-3) Brain function measurement is as in the following Table 4, which is a result converted into an evaluation in score of a visual sense, an aural sense, finger tapping, grasping power, gravity balance, cognitive function (right-and-left cognitive function, short-term memory). The short-term memory and the right-and-left cognitive function should attract attention. FIG. 16 is a graphical representation showing the mean before and after hydrogen aspiration for 17 subjects as in FIGS. 12-15. The “remarkable improvement” in the short-term memory and the right-and-left cognitive function by the hydrogen suctioning was suggested from this Table 4 and FIG. 16.

TABLE 4

| | Visual | | | Aural | | | Skin sensibility | | | Finger motion | | |
|---|---|---|---|---|---|---|---|---|---|---|---|---|
| | function | | | function | | | function | | | function | | |
| Subject | Before | After | Difference | Before | After | Difference | Before | After | Difference | Before | After | Difference |
| 1 | 6 | 6 | ±0 | 6 | 2 | +1 | 7 | 7 | ±0 | 4 | 7 | +3 |
| 2 | 2 | 5 | +3 | 6 | 6 | ±0 | 6 | 6 | ±0 | 3 | 3 | ±0 |
| 3 | 0 | 2 | +2 | 3 | 5 | +2 | 0 | 0 | ±0 | 1 | 2 | +1 |
| 4 | 0 | 1 | +1 | 3 | 4 | +1 | 4 | 4 | ±0 | 4 | 5 | +1 |
| 5 | 0 | 1 | +1 | 2 | 4 | +2 | 2 | 2 | ±0 | 2 | 4 | +2 |
| 6 | 0 | 2 | +2 | 6 | 6 | ±0 | 6 | 6 | ±0 | 4 | 5 | +1 |
| 7 | 4 | 4 | ±0 | 6 | 6 | ±0 | 6 | 8 | −1 | 5 | 7 | +2 |
| 9 | 0 | 0 | ±0 | 0 | 0 | ±0 | 0 | 2 | +2 | 3 | 5 | +2 |
| 11 | 0 | 1 | +1 | 0 | 0 | ±0 | 0 | 0 | ±0 | 4 | 4 | ±0 |
| 12 | 3 | 3 | ±0 | 6 | 6 | ±0 | 6 | 6 | ±0 | 5 | 5 | ±0 |
| 13 | 2 | 3 | +1 | 5 | 6 | +1 | 6 | 4 | −2 | 3 | 1 | −2 |
| 14 | 1 | 1 | ±0 | 1 | 4 | +3 | 0 | 0 | ±0 | 1 | 5 | +4 |
| 16 | 1 | 3 | +2 | 6 | 6 | ±0 | 6 | 5 | −1 | 4 | 4 | ±0 |
| 17 | 3 | 4 | +1 | 6 | 6 | ±0 | 1 | 4 | +3 | 3 | 5 | +2 |
| 18 | 4 | 5 | +1 | 6 | 6 | ±0 | 6 | 6 | ±0 | 6 | 7 | +1 |
| 20 | 4 | 4 | ±0 | 6 | 6 | ±0 | 5 | 6 | +1 | 10 | 8 | −2 |
| 21 | 0 | 1 | +1 | 3 | 2 | −1 | 3 | 4 | +1 | 3 | 3 | ±0 |
| Ave. | 1.8 | 2.7 | +0.9 | 4.2 | 4.7 | +0.5 | 3.8 | 3.9 | +0.2 | 3.8 | 4.7 | +0.9 |
| | Attitude | | | Knee motion | | | Right-and-left cognitive | | | Short-term | | |
| | function | | | function | | | function | | | memory | | |
| Subject | Before | After | Difference | Before | After | Difference | Before | After | Difference | Before | After | Difference |
| 1 | 10 | 10 | ±0 | 0 | 0 | ±0 | 0 | 8 | +8 | 3 | 9 | +6 |
| 2 | 6 | 10 | +4 | 10 | 8 | −2 | 0 | 9 | +9 | 0 | 8 | +8 |
| 3 | 0 | 0 | ±0 | 9 | 6 | −3 | 0 | 8 | +8 | 0 | 9 | +9 |
| 4 | 10 | 10 | ±0 | 0 | 0 | ±0 | 0 | 7 | +7 | 0 | 6 | +6 |
| 5 | 10 | 10 | ±0 | 0 | 5 | +5 | 1 | 7 | +6 | 0 | 7 | +7 |
| 6 | 10 | 10 | ±0 | 0 | 10 | +10 | 0 | 9 | +9 | 0 | 8 | +8 |
| 7 | 10 | 10 | ±0 | 10 | 10 | ±0 | 0 | 7 | +7 | 1 | 6 | +5 |
| 9 | 7 | 10 | +3 | 0 | 0 | ±0 | 0 | 6 | +6 | 0 | 7 | +7 |
| 11 | 0 | 0 | ±0 | 10 | 10 | ±0 | 1 | 6 | +5 | 0 | 5 | +5 |
| 12 | 10 | 7 | −3 | 10 | 10 | ±0 | 3 | 8 | +5 | 0 | 9 | +9 |
| 13 | 10 | 0 | −10 | 8 | 10 | +2 | 0 | 9 | +9 | 6 | 9 | +3 |
| 14 | 10 | 10 | ±0 | 7 | 0 | −7 | 0 | 9 | +9 | 0 | 9 | +9 |
| 16 | 10 | 10 | ±0 | 0 | 0 | ±0 | 0 | 7 | +7 | 0 | 8 | +8 |
| 17 | 10 | 10 | ±0 | 0 | 0 | ±0 | 0 | 8 | +8 | 0 | 8 | +8 |
| 18 | 10 | 7 | −3 | 1 | 9 | +8 | 0 | 8 | +8 | 0 | 9 | +9 |
| 20 | 10 | 10 | ±0 | 0 | 0 | ±0 | 0 | 8 | +8 | 0 | 8 | +8 |
| 21 | 10 | 10 | ±0 | 10 | 10 | ±0 | 5 | 7 | +2 | 0 | 6 | +6 |
| Ave. | 8.4 | 7.9 | −0.5 | 4.4 | 5.2 | +0.8 | 0.8 | 7.7 | +7.1 | 0.8 | 7.7 | +7.1 |

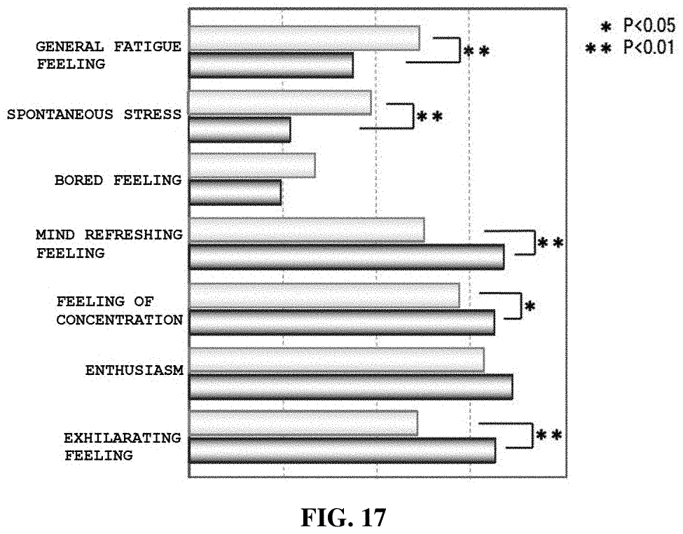

The result of 2-4) Multilateral emotional state scale is shown in FIG. 17. FIG. 17 is a graphical representation showing the mean before and after hydrogen aspiration for 17 subjects as in FIGS. 12 to 16. This result showed that the fatigue feeling and spontaneous stress decreased by the hydrogen suctioning, while feelings of refreshing, concentration, and exhilarating were significantly raised.

### EXPLANATION OF REFERENCE NUMERALS

100 Electrolytic hydrogen gas suction tool
1 Body cover
2 Mixer
13 Hydrogen passing member
13a Film material (breathable impermeable material)
14 Ampule section
15 Lidding member
16 Metal materials
17 Container body
18 Aqueous solution
19 Closure member
20 Hydrogen
22 Non-reaction portion
24 Metallic particle layers
40 Convex shape portion
41 Thinned portion
100, 200 Hydrogen gas aspirator (Hydrogen gas generator)
102 Aspirator main body
104 Suction coat
105 Cap member
106, 206 Interconnection
108, 208 mouthpiece member
110, 210 Film packing
112 Regulating valve
113, 213 Window
114 Adjustment port
116 Cartridge
117, 217 Gaps
118 O-ring
300 Wearable handheld
310 Mobile Handsets
320 Calculation means
330 Portable gas generator (hydrogen gas extractor)
340 Server

1. A healthcare system comprising:
a portable gas generator capable of suctioning a selected gas among mixed gases containing hydrogen and/or oxygen, which are produced by electrolysis of an electrolyte filled in at least an electrolysis tank provided inside, for a predetermined period of time by spontaneous respiration,
wherein said mixed gas having an effect of promoting neural activity and/or circulatory activity of a living body; and
a wearable portable terminal for detecting a vital sign that is digitized by contacting and fixing a part of a body part.

2. A program for use in a healthcare system according to claim 1, comprising:
a detecting means for detecting the vital signs that are digitized by contacting and fixing the wearable portable terminal to a part of a human body part;
a first wireless transmitting means for wirelessly transmitting the vital signs to the outside; and
a calculating means for calculating a required gas and gas discharge amount and/or time that are set in advance in accordance with each of the vital signs detected by the wearable portable terminal, and further comprising any one of the following (1), (2) and (3):
(1) second wireless transmission means for wirelessly transmitting the required gas discharged from the gas generator and the gas discharge amount and/or time to the outside;
(2) control signal transmitting means for transmitting a control signal based on the calculated required gas and gas discharge amount and/or time, and control means for receiving the control signal to control the power supply of the portable gas generator; and
(3) second wireless transmission means for wirelessly transmitting the required gas and the gas discharge amount and/or time discharged from the gas generator to the outside, control signal transmission means for transmitting a control signal based on the calculated required gas and gas discharge amount and/or time, and control means for receiving the control signal and controlling the power supply of the portable gas generator.

3. The program of claim 2, wherein the wearable portable terminal comprises the calculating means.

4. The portable gas generator according to claim 1, comprising:
a body cover member having a battery, a control board for controlling power supply from the battery; and
a pair of anode electrodes energized or blocked an anode and cathode of battery by the control board;
a water reservoir electrolysis tank detachably attached to the body cover member, the pair of anode electrodes are inserted into the interior in a mounted state;
a nozzle portion having a through hole; and
a mixing portion having a flow path for fluidly connecting nozzle portion and an end portion of the electrolysis tank and introducing atmosphere.

5. A portable gas-generator as claimed in claim 2, comprising:
a body cover member having a battery, a control board for controlling power supply from the battery; and
a pair of anode electrodes energized or blocked an anode and cathode of battery by the control board;
a water reservoir electrolysis tank detachably attached to the body cover member, the pair of anode electrodes are inserted into the interior in a mounted state;
a nozzle portion having a through hole; and
a mixing portion having a flow path for fluidly connecting nozzle portion and an end portion of the electrolysis tank and introducing atmosphere.
